(19) United States
(12) Patent Application Publication
(10) Pub. No.: US 2005/0164184 A1
(43) Pub. Date: Jul. 28, 2005

(54) Hybridization portion control oligonucleotide and its uses

(76) Inventor: Jong-Yoon Chun, Seoul (KR)

Correspondence Address: NIXON & VANDERHYE, PC, 901 NORTH GLEBE ROAD, 11TH FLOOR, ARLINGTON, VA, 22203, US

(21) Appl. No.: 10/498,108

(22) PCT Filed: Nov. 4, 2002
(86) PCT No.: PCT/KR02/02051

(30) Foreign Application Priority Data
Dec. 8, 2001 (WO) PCT/KR01/02133
May 1, 2002 (WO) PCT/KR02/00816

(51) Int. Cl. C12Q 1/68; C07H 21/04

(52) U.S. Cl. 435/6; 536/24.3; 536/25.32

(57) ABSTRACT

The present invention relates to an oligonucleotide for analyzing a target nucleotide sequence by hybridization and its applications. The oligonucleotide has the following general structure: 5′-Xₚ—Y_{q}-Zᵣ-3′ or 5′-Zᵣ—Y_{q}-Xₚ-3′ wherein Xₚ represents a first hybridization portion having a specific hybridizing nucleotide sequence substantially complementary to said target nucleotide sequence in said sample nucleic acid to hybridize therewith; Y_{q} represents a regulator portion comprising at least two universal bases or non-discriminatory base analogs; Zᵣ represents a second hybridization portion having a pre-selected arbitrary nucleotide sequence; p, q and r represent the number of nucleotides; and X, Y and Z is deoxyribonucleotide or ribonucleotide.

### BACKGROUND OF TE INVENTION

1. Field of the Invention

The present invention is in the field of nucleic acid hybridization for analyzing a selected nucleotide sequence of a sample nucleic acid. More specifically, the present invention relates to a hybridization portion control oligonucleotide with a novel structure which has dual functions for generating specific hybridization and verifying hybridization results and to its applications.

2. Description of the Related Art

DNA hybridization, in which a DNA strand binds its complement to form a duplex structure, is a fundamental process in molecular biology. The formation of duplexes is affected by ionic strength, base composition, length of fragment to which the nucleic acid has been reduced, the degree of mismatching and the presence of denaturing agents. In principle, any nucleic acid—double-stranded DNA, single-stranded DNA, oligonucleotides, mRNA and RNA—can act as a probe. The choice is determined by the purpose of the experiment. In general, the oligonucleotide probe is used to form perfectly-matched duplexes with a target sequence. The oligonucleotide hybridization assay is based on the following general scheme: the probe or target is labeled (e.g., with a radioactive isotope, a fluorescent dye or a reactive compound), the nucleic acids are placed under hybridization conditions following hybridization, the non-hybridized labeled material is removed and the remaining label is quantitated.

A method to enhance the distinction between exact duplexes and duplexes with one or more mismatched base pairs would be a very useful tool in specific nucleic acid sequence determination and clearly be valuable in clinical diagnosis, genetic research and forensic laboratory analysis. For example, Wallace and coworkers showed that sequence differences as subtle as a single base change are sufficient to enable discrimination of short (e.g., 14-mer) oligomers, and demonstrated the utility in the molecular analysis of point mutation in the β-globin gene (Wallace et al., 1981; Conner et al., 1983).

In spite of the power of oligonucleotide hybridization to correctly identify a complementary strand, it does face limitations. Hybrids containing oligonucleotides are much less stable than hybrids of long nucleic acids. This is reflected in lower melting temperature. The instability of the hybrids is one of the most important factors to be considered when designing oligonucleotide hybridization. The stability difference between a perfectly matched complement and a complement mismatched at only one base can be quite small, corresponding to as little as 0.5° C. difference in their Tₘs (duplex melting temperature) (Tibanyenda et al., 1984; Werntges et al., 1986). The shorter the oligomer of interest (permitting identification of a complementary strand in a more complex mixture), the stronger the effect of a single-base mismatch on overall duplex stability. However, the disadvantage of using such short oligonucleotides is that they hybridize weakly, even to a perfectly complementary sequence, and thus must be used under conditions of reduced stringency. Thus, the need remains for a method of modifying short oligonucleotides which are more stable even under conditions of high stringency, such that the specificity of short oligonucleotide hybridization will be improved under such high stringency enough to achieve single nucleotide mismatch discrimination.

There have been many efforts to improve the specificity of oligonucleotide hybridization. A method for chemically modifying bases of DNA for high-sensitivity hybridization (Azhikina et al., Proc. Natl. Acad. Sci., USA, 90: 11460-11462 (1993)) and a method in which the washing after the hybridization is conducted at low temperatures for a long period time to enhance the ability of discriminating the mismatch (Drmanac et al., DNA and Cell Biology, 9: 527-534 (1990)) have been proposed. Recently, another method has been introduced for increasing the discrimination of single nucleotide polymorphisms (SNPs) in DNA hybridization by means of artificial mismatches (Guo et al., Nature Biotechnology, 15: 331-5(1997)). In addition, many U.S. Patents including U.S. Pat. Nos. 6,077,668, 6,329,144, 6,140,054, 6,350,580, 6,309,824, 6,342,355 and 6,268,128 disclose the probe for hybridization and its applications. Although the improved approaches to each method has been continuously introduced, all these methods and techniques involving oligonucleotide hybridization could not be completely free from the limitations and problems from non-specificity of oligonucleotide hybridization.

Furthermore, there is still possibility that artificial factors such as the failures of spotting and immobilization of oligonucleotide on substrate and establishment of optimal hybridization conditions would affect the negative data of hybridization; especially the effect of erroneous results is more vulnerable to the results generated from high-throughput screening method. Such artificial factors inherent to spotting and hybridization are main practical drawbacks in oligonucleotide-based DNA microarrays. The ability to use oligonucleotide which itself has the function for verification of the hybridization results would therefore be beneficial in oligonucleotide-based DNA microarrays.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

Endeavoring to resolve the problems of such conventional probe and hybridization methods, the present inventor has developed a novel oligonucleotide that can permit hybridization with much higher specificity and its unlimited applications in all fields of hybridization-based technology.

Accordingly, it is an object of this invention to provide an oligonucleotide for analyzing a target nucleotide sequence in a sample nucleic acid by hybridization.

It is another object of this invention to provide a method for detecting the presence of a target nucleotide sequence in a sample nucleic acid by hybridization.

It is still another object of this invention to provide a method for identifying a nucleotide variation in a target nucleotide sequence of a sample nucleic acid.

It is further object of this invention to provide a kit for carrying out a hybridization.

It is still further object of this invention to provide a kit for identifying a nucleotide variation in a target nucleic acid of a sample nucleic acid.

Other objects and advantages of the present invention will become apparent from the detailed description to follow taken in conjugation with the appended claims and drawings.

### BRIEF DESCRIPTION OF THE DRAWINGS

FIG. 1 is an autoradiograph evaluating the hybridization specificity of the present oligonucleotide. In all panels, the wild-type allele-specific oligonucleotide (lane 1) and the mutant-type allele-specific oligonucleotide (lane 2) are immobilized on nylon membrane. Panel A demonstrates that the oligonucleotides used in the experiment are spotted equally on each membrane. In panel B, the results of the first hybridization for the mutant-type genomic DNA fragment are shown. Panel C shows the results of the second hybridization to verify the results from the first hybridization.

FIG. 2 is an autoradiograph showing the results from the hybridization reactions for the wild-type genomic DNA fragment using the present oligonucleotide. The wild-type allele-specific oligonucleotide (lane 1), the mutant-type allele-specific oligonucleotide (lane 2) and the negative control oligonucleotide (lane 3) are immobilized on nylon membrane. In panel B, the results of the first hybridization for the wild-type genomic DNA fragment are shown. Panel C represents the results of the second hybridization to verify the results from the first hybridization.

FIG. 3 is an autoradiograph to represent the results from the hybridization reactions for the mutant-type genomic DNA fragment using the present oligonucleotide. The descriptions of lanes and panels are the same as those of FIG. 2.

FIG. 4 is an autoradiograph to show the results from the hybridization reactions for the heterozygous-type genomic DNA fragment using the present oligonucleotide. The descriptions of lanes and panels are the same as those of FIG. 2.

### DETAILED DESCRIPTION OF THIS INVENTION

The present invention is generally directed to an oligonucleotide having dual functions for generating specific hybridization and verifying hybridization results quantitatively. The oligonucleotide of this invention, named hybridization portion control oligonucleotide (hereinafter referred to as “HPC oligonucleotide”), ensures a very specific hybridization reaction to a target nucleotide sequence, such that a variety of analyses using hybridization can be performed with higher reliability.

The principle of the HPC oligonucleotide is based on its novel structure having (i) a first hybridization portion having a nucleotide sequence substantially complementary to target nucleotide sequence, (ii) a second hybridization portion having a pre-selected arbitrary nucleotide sequence, and (iii) a regulator portion comprising at least two universal bases or non-discriminatory analogues positioned between the first hybridization portion and the second hybridization portion.

The present HPC oligonucleotide allows the first and second hybridization portions to be involved in two independent hybridizations as a first hybridization and second hybridization, respectively. The presence of universal base or non-discriminatory base residue group in the HPC oligonucleotide permits only the first hybridization portion to be hybridized with target nucleotide sequence of interest at a first hybridization. Furthermore, the second hybridization portion serves as a universal hybridizing site at a second hybridization for quantitative verification of the results from the first hybridization reaction. Therefore, solely using this dual functional HPC oligonucleotide, a specific hybridization with target nucleotide sequence and a quantitative verification of the first hybridization results can be accomplished.

For these reasons, the HPC oligonucleotide of the present invention is fundamentally different from the conventional oligonucleotides in terms of the way for improving a specificity of oligonucleotide hybridization under a certain stringency conditions as well as quantitative verification of hybridization results.

The HPC oligonucleotide of this invention is significantly effective and widely accessible to nucleic acid hybridization-based applications. In addition, various problems associated with hybridization specificity in conventional oligonucleotides can be fundamentally solved by the HPC oligonucleotide of the present invention. The features and advantages of the present HPC oligonucleotide are summarized as follows:
(a) since a regulator portion of the present oligonucleotide is composed of at least two universal base or non-discriminatory analogue which has lower Tₘ than other portions in the present oligonucleotide due to its weaker hydrogen bonding interactions in base pairing, it is not favorable in hybridization with the target nucleic acid under the conditions that the first hybridization portion of the HPC oligonucleotide is hybridized with the target nucleic acid. Thus, the presence of an universal base residue group between the first and second hybridization portions forms a boundary between these portions, which restricts a hybridization portion of the oligonucleotide to the first hybridization portion under such conditions that the first hybridization portion is hybridized with a target nucleotide sequence so that the second hybridization portion may be completely excluded from the hybridization reaction between the first hybridization portion and its target nucleotide sequence. That is, the regulator portion is capable of interrupting the hybridization of the second hybridization portion when the first hybridization portion is hybridized with the target nucleotide sequence so that the hybridization specificity of the first hybridization portion can be increased. Therefore, the hybridization sequence of the oligonucleotide can be precisely controlled, which makes it possible to design an oligonucleotide capable of having a desired number of hybridization sequence. It is particularly useful when a hybridization portion of an oligonucleotide has to be limited (e.g., single nucleotide polymorphism (SNP) genotyping, DNA microarray screening and detection of differentially expressed genes);
(b) the second hybridization portion not complementary to a target nucleotide sequence leaves the first hybridization portion free to hybridize with its target nucleotide sequence when the HPC oligonucleotide of this invention is bound to a substrate such as nylon membrane and glass, thereby increasing hybridization strength (efficiency) of the first hybridization portion;
(c) the increased hybridization strength (efficiency) of the first hybridization portion allows hybridization reaction to be performed under higher stringent conditions which includes higher hybridization and washing temperatures, so that the hybridization specificity of the first hybridization portion is increased;
(d) the above-mentioned features of the present HPC oligonucleotide leads to the dramatic enhancement of the hybridization specificity so that even one mismatch throughout the hybridized duplex may be discriminated from complete match; thus, the present HPC oligonucleotide is particularly useful for the identification of a nucleotide variation in a target nucleic acid, including, for example, single nucleotide polymorphisms and point mutations; and provides an oligonucleotide with a high tolerance in “search parameters” for probe design such as oligonucleotide length, hybridization temperature and GC content; and
(e) the second hybridization portion also permits the verification of the first hybridization results, which can exclude the first hybridization data from erroneous results due to artificial effects such as the failures of immobilization of oligonucleotide on substrate and establishment of optimal hybridization conditions.
   Principle of HPC Oligonucleotide of this Invention

In one aspect of this invention, there is provided a HPC oligonucleotide for analyzing a target nucleotide sequence in a sample nucleic acid by hybridization. The HPC oligonucleotide has the following general structure:
5′Xₚ—Y_{q}-Zᵣ-3′ or 5′-Zᵣ-Y_{q}—Xₚ-3′
wherein Xₚ represents a first hybridization portion having a specific hybridizing nucleotide sequence substantially complementary to the target nucleotide sequence in the sample nucleic acid to hybridize therewith; Y_{q} represents a regulator portion comprising at least two universal bases or non-discriminatory base analogs; Zr represents a second hybridization portion having a pre-selected arbitrary nucleotide sequence; p, q and r represent the number of nucleotides; and X, Y and Z is deoxyribonucleotide or ribonucleotide.

The principle of the present HPC oligonucleotide is based on its novel structure having the first hybridization and second hybridization portions separated by a regulator portion comprising at least two universal bases or non-discriminatory bases and the effect of the regulator portion on the first hybridization and second hybridization portions. The introduction of the regulator portion between the first and second hybridization portions, comprising at least two universal bases or non-discriminatory bases, acts as a main factor that is responsible for the improvement of hybridization specificity.

The term, “sample” in conjunction with nucleic acid refers to any substance containing or presumed to contain a nucleic acid of interest (a target nucleotide sequence) or which is itself a nucleic acid containing or presumed to contain a target nucleotide sequence of interest. The term “sample” thus includes a sample of nucleic acid (genomic DNA, cDNA, RNA), cell, organism, tissue, fluid, or substance including but not limited to, for example, plasma, serum, spinal fluid, lymph fluid, synovial fluid, urine, tears, stool, external secretions of the skin, respiratory, intestinal and genitourinary tracts, saliva, blood cells, tumors, organs, tissue, samples of in vitro cell culture constituents, natural isolates (such as drinking water, seawater, solid materials) and microbial specimens. The term “nucleic acid” is a deoxyribonucleotide or ribonucleotide polymer in either single or double-stranded form, including known analogs of natural nucleotides unless otherwise indicated. Thus, the oligonucleotide of this invention can be employed in hybridization using double-stranded or preferably, single gDNA, cDNA or mRNA as a sample nucleic acid. There is no intended distinction between the terms “nucleic acid” and “nucleotide”, and these terms will be used interchangeably.

The term “oligonucleotide” as used herein refers to a linear oligomer of natural or modified monomers or linkages, including deoxyribonucleotides, ribonucleotides and the like, capable of specifically hybridizing with a target nucleotide sequence, whether occurring naturally or produced synthetically. The oligonucleotide is preferably single stranded for maximum efficiency in hybridization. Preferably, the oligonucleotide is an oligodeoxyribonucleotide. The HPC oligonucleotide of this invention can be comprised of naturally occurring dNMP (i.e., dAMP, dGMP, dCMP and dTMP), nucleotide analogs or nucleotide derivatives. The oligonucleotide can also include ribonucleotides. For example, the HPC oligonucleotide of this invention may include nucleotides with backbone modifications such as peptide nucleic acid (PNA) (M. Egholm et al., Nature, 365: 566-568(1993)), phosphorothioate DNA, phosphorodithioate DNA, phosphoramidate DNA, amide-linked DNA, MM-linked DNA, 2′-O-methyl RNA, alpha-DNA and methylphosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine and diaminopurine.

The term “portion” used herein in conjunction with the HPC oligonucleotide of this invention refers to a nucleotide sequence separated by the regulator portion. The term “first hybridization portion” with reference to the present HPC oligonucleotide refers to a portion having a specific hybridizing nucleotide sequence substantially complementary to a target nucleotide sequence in a sample nucleic acid to hybridize therewith. The first hybridization portion may be located at either 3′- or 5′-end portions of the HPC oligonucleotide. The term “second hybridization portion” with reference to the present HPC oligonucleotide refers to a portion having a pre-selected arbitrary nucleotide sequence. In addition, the second hybridization portion may be located at either 3′- or 5′-end portions of the present HPC oligonucleotide. The term “arbitrary” nucleotide sequence is used herein to mean the nucleotide sequence that is chosen without knowledge of the sequence of the target nucleic acids to be hybridized.

The term “hybridization” with reference to the HPC oligonucleotide refers to the formation of a double-stranded nucleic acid by base-pairing between a nucleic acid sequence and a portion of the HPC oligonucleotide. As used herein, a “probe” refers to a single-stranded nucleic acid molecule comprising a portion or portions that are substantially complementary to a target nucleotide sequence. The first hybridization portion of the present oligonucleotide has a nucleotide sequence substantially complementary to a target nucleotide sequence in a sample nucleic acid. The term “substantially complementary” with reference to oligonucleotide is used herein to mean that the oligonucleotide is sufficiently complementary to hybridize selectively to a target nucleic acid sequence under the designated hybridization conditions. Therefore, this term has a different meaning from “perfectly complementary” or related terms thereof. It will be appreciated that the first hybridization portion of the HPC oligonucleotide may have one or more mismatches to a target nucleotide sequence to an extent that the desired hybridization specificity may be accomplished. The term “specificity” with referring to hybridization means the fidelity of hybridization to be made between completely or perfectly complementary bases. Most preferably, the first hybridization portion of the present oligonucleotide has a nucleotide sequence perfectly complementary to a target nucleotide sequence in a sample nucleic acid, i.e., no mismatches.

The first hybridization portion of the present HPC oligonucleotide may have a wide variety of nucleotide sequences depending on its applications as well as a target nucleotide sequence. For example, where the present HPC oligonucleotide is applied to a method for identifying a nucleotide variation in a target nucleotide sequence of a sample nucleic acid, its first hybridization portion has a nucleotide sequence comprising a nucleotide complementary to the corresponding nucleotide of a nucleotide variation. Furthermore, where the present HPC oligonucleotide is employed in differential display, an arbitrary sequence substantially complementary to a site in a cDNA from an mRNA is composed of the first hybridization portion of the present HPC oligonucleotide; in identification of conserved homology segment, the first hybridization portion of the present HPC oligonucleotide has a nucleotide sequence substantially complementary to a consensus sequence found in a gene family or degenerate sequence selected from a plurality of combinations of nucleotides encoding a predetermined amino acid sequence.

The regulator portion comprising at least two universal bases or non-discriminatory base analogs is responsible for higher hybridization specificity of the present HPC oligonucleotide. The term “universal base or non-discriminatory base analog” used herein refers to one capable of forming base pairs with each of the natural DNA/RNA bases with little discrimination between them.

It has been widely known that nucleotides at some ambiguous positions of degenerate oligonucleotides have been replaced by universal base or a non-discrimatory analogue such as deoxyinosine (Ohtsuka, E. et al., J. Biol. Clem. 260: 2605-2608(1985); Sakanari, J. A. et al., Proc. Natl. Acad. Sci. 86: 4863-4867(1989)), 1-(2′-deoxy-beta-D-ribofuranosyl)-3-nitropyrrole (Nichols, R. et al., Nature 369: 492493(1994)) and 5-nitroindole (Loakes, D. and Brown, D. M. Nucleic Acids Res. 22: 4039-4043(1994)) for solving the design problems associated with the degenerate oligonucleotides because such universal bases are capable of non-specifically base pairing with all four conventional bases. However, there has not been any report that this universal base or a non-discriminatory analogue such as deoxyinosine, 1-(2′-deoxy-beta-D-ribofuranosyl)-3-nitropyrrole and 5-nitroindole is used as a regulator portion which is capable of controlling a hybridization portion in an oligonucleotide. As a unique portion of the HPC oligonucleotide, the regulator portion separates the two portions functionally or structurally.

The presence of universal base such as deoxyinosine, 1-(2′-deoxy-beta-D-ribofuranosyl)-3-nitropyrrole and 5-nitroindole in a HPC oligonucleotide generates a lower melting temperature due to its weaker hydrogen bonding interactions in base pairing. As an extension of this theory, the present inventor has induced that the introduction of the universal bases between the first and second hybridization portions of a HPC oligonucleotide could generate a region which has lower melting temperature, form a boundary to each of the first and second hybridization portions of the HPC oligonucleotide, and affect the hybridization of each portion. This theory provides the basis of the HPC oligonucleotides of this invention.

In a preferred embodiment, the present oligonucleotide contains at least three universal bases or non-discriminatory base analogs between the first hybridization and second hybridization portion sequences, more preferably, at least 4 universal bases or non-discriminatory base analogs. Advantageously, the universal base residues between the first and second hybridization portion sequences can be up to 15 residues in length. According to one embodiment, the present HPC oligonucleotide contains 2-15 universal bases or non-discriminatory base analogs. Most preferably, the universal bases between the first and second hybridization portion sequences are about 5 residues in length. With reference to the optimum number of universal base, i.e., 5 residues, the minimum number of universal base residues between the first and second hybridization portions of the present HPC oligonucleotide is preferred in order to interrupt the hybridization of the second hybridization portion to a target nucleotide sequence under certain hybridization condition. It is very likely that the length of universal base in the sequence (8-10 bases) does not make a significant difference on its own function in the present HPC oligonucleotide.

According to a preferred embodiment, the universal base or non-discriminatory base analog in the regulator portion of the HPC oligonucleotide includes deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitroindole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-0-methoxyethyl inosine, 2′0-methoxyethyl nebularine, 2′-0-methoxyethyl 5-nitroindole, 2′-0-methoxyethyl 4-nitro-benzimidazole, 2′-0-methoxyethyl 3-nitropyrrole and combinations thereof, but not limited to. More preferably, the universal base or non-discriminatory base analog is deoxyinosine, 1-(2′-deoxy-beta-D-ribofuranosyl)-3-nitropyrrole or 5-nitroindole, most preferably, deoxyinosine.

Preferably, the overall length of the HPC oligonucleotide is determined from desired specificity of hybridization and the number of nucleotides that are required to hybridize to a target nucleic acid.

The length of each portion of the present HPC oligonucleotide may vary and depend in part on the objective of each application using the present HPC oligonucleotide. In a preferred embodiment, the first hybridization portion of the present HPC oligonucleotide is at least 6 nucleotides in length, which is considered a minimal requirement of length for oligonucleotide hybridization. More preferably, the first hybridization portion sequence is from 10 to 50 nucleotides and can be up to 100 nucleotides in length.

As discussed above, since the second hybridization portion of the HPC oligonucleotide is completely excluded from the hybridization between the first hybridization portion and its target nucleotide sequence, the selection of its nucleotide sequence is not subject to restriction. That is, even when the nucleotide sequence of the second hybridization portion is more or less complementary to a site in a sample nucleic acid (e.g. a site adjacent to target nucleotide sequence of the first hybridization portion), the hybridization specificity of the first hybridization portion with its target nucleotide sequence is not practically reduced. This is one of the advantages of the HPC oligonucleotide. Preferably, the second hybridization portion of the HPC oligonucleotide comprises a pre-selected arbitrary nucleotide sequence substantially not complementary to any site on the sample nucleic acid.

The second hybridization portion leaves the first hybridization portion free to hybridize with its target nucleotide sequence when the HPC oligonucleotide is bound to a substrate such as nylon membrane and glass, thereby increasing hybridization strength (efficiency) of the first hybridization portion, which is consistent with a spacer effect proposed by Saiki and coworkers (Randall K. Saiki, et al., PNAS USA, 86: 6230-6234(1989)). They showed that homopolymer tails applied to oligonucleotides improve hybridization efficiency by increasing the distance between the nylon membrane and the tailed oligonucleotide probe. Therefore, the increased hybridization strength (efficiency) of the first hybridization portion allows hybridization reaction to be performed under higher stringent conditions that include higher hybridization and washing temperatures so that the hybridization specificity of the first hybridization portion is highly increased. In addition, the second hybridization portion permits verification of the hybridization results between the first hybridization portion and its target nucleotide sequence. Such verification may obtain the reliable results solely from the hybridization between the first hybridization portion and its target nucleotide sequence.

In a preferred embodiment, the second hybridization portion of the present HPC oligonucleotide contains at least 15 nucleotides in length, which is considered a minimal requirement of length for hybridization. Preferably, the second hybridization portion sequence can be up to 100 nucleotides in length. More preferably, the second hybridization portion sequence is from 20 to 30 nucleotides in length. It is preferred that the second hybridization portion is longer than the first hybridization.

The pre-selected arbitrary nucleotide sequence of the second hybridization portion may be any defined or pre-selected deoxyribonucleotide, ribonucleotide, or mixed deoxyribonucleotide sequence which contains a particular sequence of natural or modified nucleotides.

According to one embodiment of the present invention, some modifications in the present HPC oligonucleotide can be made unless the modifications abolish the advantages of the HPC oligonucleotide, i.e., improvement in hybridization specificity. These modifications, i.e., labels can provide a detectable signal for indicating hybridization, and which may be linked to the HPC oligonucleotide. Suitable labels include, but not limited to, fluorophores, chromophores, chemiluminescers, magnetic particles, radioisotopes, mass labels, electron dense particles, enzymes, cofactors, substrates for enzymes and haptens having specific binding partners, e.g., an antibody, streptavidin, biotin, digoxigenin and chelating group. Labels can provide signals detectable by fluorescence, radioactivity, colorimetry, gravimetry, X-ray diffraction or absorption, magnetism, enzymatic activity, mass spectrometry, binding affinity, hybridization radiofrequency, nanocrystals and the like. The HPC oligonucleotide of the invention is labeled at the 5′-end, the 3′-end or internally. The label can be direct, i.e. a dye, or indirect, i.e. biotin, digoxin, alkaline phosphatase, horse radish peroxidase, etc.

According to one embodiment of the present invention, the present HPC oligonucleotide can be immobilized on an insoluble carrier. Examples of the insoluble carrier include a nitrocellulose or nylon filter, a glass plate, silicone and fluorocarbon supports. The HPC oligonucleotides may be immobilized, by a number of methods known to those skilled in the art, such as laser-activated photodeprotection attachment through a phosphate group using reagents such as a nucleoside phosphoramidite or a nucleoside hydrogen phosphorate.

The HPC oligonucleotides immobilized may be organized into an array or arrays for certain applications. Hydrophobic partitions may be used to separate HPC oligonucleotides or arrays of HPC oligonucleotides. Arrays may be designed for various applications (e.g. mapping, partial sequencing, sequencing of targeted regions for diagnostic purposes, mRNA sequencing and large scale sequencing). A specific microarray may be designed to be dedicated to a particular application by selecting a combination and arrangement of various HPC oligonucleotides with distinct sequences on an insoluble carrier such as a glass plate.

According to the preferred embodiment of this invention, the HPC oligonucleotide is applied to a method for detecting the presence of a target nucleotide sequence by hybridization, i.e., used as a probe specific for a target nucleotide sequence. More preferably, the oligonucleotide of this invention provides a very powerful tool in a method for detecting the presence of a target nucleotide sequence (even a nucleotide) requiring very high hybridization specificity.

The hybridization reaction using the HPC oligonucleotide may be performed in two separate hybridizations, i.e. the first and second hybridizations, respectively. The first hybridization portion of the HPC oligonucleotide is involved in a first hybridization with a target nucleic acid and the second hybridization portion is involved in a second hybridization for demonstrating the verification of the results from the hybridization of the first hybridization portion.

In another aspect of this invention, there is provided a kit for carrying out a hybridization, which comprises an HPC oligonucleotide or an HPC oligonucleotide set according to the present invention. According to one embodiment of this invention, this kit further comprises an oligonucleotide having a nucleotide sequence complementary to the second hybridization portion of the oligonucleotide. In addition, the present kit further comprises an oligonucleotide without the regulator portion but comprising the same nucleotide sequence as the present oligonucleotide. The present kit may optionally include the reagents required for hybridization reaction such as buffers. Optimal amounts of reagents to be used in a given reaction can be readily determined by the skilled artisan having the benefit of the current disclosure. The kits, typically, are adapted to contain in separate packaging or compartments the constituents afore-described.

The present HPC oligonucleotide can be applied to a variety of hybridization-based technologies. Representative examples to prove the effect of the present oligonucleotide are: (i) detection of a target nucleotide sequence; (ii) identification of nucleotide variation in a target nucleotide sequence; (iii) sequencing by hybridization (Genomics, 13:1378(1992)); (iv) identification of differences in nucleic acid levels between two or more nucleic acid samples (see U.S. Pat. No. 5,143,854); (v) in situ hybridization (Young, W. S., In Situ Hybridization: A Practical Approach. New York: Oxford University Press; 33-44(1992)); (vi) diagnosis of infectious diseases and hereditary diseases; and (vii) mapping of giant genomic DNA.

Application to General Hybridization

In still another aspect of this invention, there is provided a method for detecting the presence of a target nucleotide sequence in a sample nucleic acid by hybridization, wherein the method comprises the steps of: (a) performing a first hybridization using a first HPC oligonucleotide described above having at its first hybridization portion a specific hybridizing nucleotide sequence substantially complementary to the target nucleotide sequence to hybridize therewith under conditions in which the first hybridization portion of the first oligonucleotide is to be hybridized to the target nucleotide sequence; and (b) detecting the presence or absence of the target nucleotide sequence substantially complementary to the first hybridization portion of the first oligonucleotide in the sample nucleic acid through a signal indicative of the hybridization between the target nucleotide sequence and the first hybridization portion.

Since the present method employs the HCP oligonucleotide, the common descriptions between them are omitted in order to avoid the complexity of this specification leading to undue multiplicity.

According to the present method, the steps (a) and (b) constitute the process of hybridization with target nucleotide sequence, herein referred to as “first hybridization”. Therefore, the presence or absence of a target nucleotide sequence in a sample nucleic acid can be determined through a signal indicative of the hybridization between the target nucleotide sequence and the specific portion of the first oligonucleotide. The term “first oligonucleotide” used herein refers to the oligonucleotide used in the first hybridization, which has the same structure as the HCP oligonucleotide described previously. It is preferred that the first oligonucleotide rather than target nucleotide sequence is immobilized on an insoluble carrier (substrate) described above. Preferably, the hybridization in the step (a) is performed under higher stringent conditions than used conventionally, which results in improvement of the hybridization specificity.

The regulator portion of the first oligonucleotide used is capable of restricting a hybridization portion of the first oligonucleotide with the target nucleotide sequence to the first hybridization portion. Therefore, the regulator portion of the first oligonucleotide plays a key role in enhancing a hybridization specificity of the first hybridization portion of the first oligonucleotide.

According to a preferred embodiment, this method further comprises the steps of: (c) performing a second hybridization using a second oligonucleotide having a nucleotide sequence substantially complementary to the second hybridization portion of the first oligonucleotide used in step (a) to hybridize therewith under conditions in which the second oligonucleotide is to be hybridized to the second hybridization portion sequence of the first oligonucleotide; and (d) detecting a signal indicative of the hybridization between the second hybridization portion of the first oligonucleotide and the second oligonucleotide, so that the presence or absence of the signal of step (b) is confirmed to be ascribed solely to the hybridization between the target nucleotide sequence and the first hybridization portion of the first oligonucleotide.

According to the preferred embodiment, the steps (c) and (d) constitute the process of verifying the results from the first hybridization, herein referred to as “second hybridization”. The term “second oligonucleotide” used herein refers to the oligonucleotide used in the second hybridization, which has a nucleotide sequence substantially complementary to the second hybridization portion of the first oligonucleotide.

In the present method, the suitable hybridization conditions may be routinely determined by optimization procedures. Such procedures are routinely conducted by those skilled in the art to establish protocols for use in a laboratory. For example, conditions such as temperature, concentration of components, hybridization and washing times, buffer components, and their pH and ionic strength may be varied depending on various factors such as the length and GC content of oligonucleotide and target nucleotide sequence. For instance, when a relatively short oligonucleotide is used, it is preferably that low stringent condition be adopted. The detailed conditions for hybridization can be found in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999).

Application to Identification of Nucleotide Variation

In further aspect of this invention, there is provided a method for identifying a nucleotide variation in a target nucleotide sequence of a sample nucleic acid, wherein the method comprises the steps of: (a) performing a first hybridization using a first HCP oligonucleotide described above having at its first hybridization portion a specific hybridizing nucleotide sequence substantially complementary to the target nucleotide sequence of the sample nucleic acid to hybridize therewith under conditions in which the first hybridization portion of the first oligonucleotide is to be hybridized to the target nucleotide sequence of the sample nucleic acid, wherein each of the first oligonucleotide and the target nucleotide sequence comprises an interrogation position corresponding to the nucleotide variation, whereby the first oligonucleotide including the nucleotide variation is hybridized to the target nucleotide sequence when the interrogation position is occupied by the complementary nucleotide of the first oligonucleotide to its corresponding nucleotide of the target nucleotide sequence; and (b) identifying the nucleotide variation in the target nucleotide sequence of the sample nucleic acid by detecting a signal indicative of the hybridization between the target nucleotide sequence and the first hybridization portion of the first oligonucleotide.

Since this application using the HCP oligonucleotide of this invention is carried out in accordance with the present method for detecting the presence of a target nucleotide sequence, the common descriptions between them are omitted in order to avoid the complexity of this specification leading to undue multiplicity.

According to a preferred embodiment, the method further comprises the steps of: (c) performing a second hybridization using a second oligonucleotide having a nucleotide sequence substantially complementary to the second hybridization portion of the first oligonucleotide used in the step (a) to hybridize therewith under conditions in which the second oligonucleotide is to be hybridized with the second hybridization portion sequence of the first oligonucleotide; and (d) detecting a signal indicative of the hybridization between the second hybridization portion of the first oligonucleotide and the second oligonucleotide, so that the presence or absence of the signal of step (b) is confirmed to be ascribed solely to the hybridization between the target nucleotide sequence and the first hybridization portion of the first oligonucleotide.

The sample nucleic acid used may be a short nucleotide segment including a nucleotide variation that is prepared by amplifying the corresponding nucleotide sequence of the short nucleotide segment. In addition, the sample nucleic acid may be more than one target short nucleotide segment each including a nucleotide variation which is prepared by amplifying each corresponding nucleotide sequence of more than one short nucleotide segment.

In a preferred embodiment, the nucleotide variation to be detectable is single nucleotide polymorphism or point mutation. The nucleotide variation may be contained within human nucleic acid or within nucleic acid of an organism causing an infectious disease.

The first oligonucleotide used in step (a) is an allele-specific HCP oligonucleotide which contains an interrogation position within its first hybridization portion occupied by a complementary nucleotide to the corresponding nucleotide of the nucleotide variation in a target nucleic acid. Preferably, the interrogation position of the first oligonucleotide is in the middle of its first hybridization portion. In a more preferred embodiment, the interrogation position of the allele-specific HCP oligonucleotide is within about 10 bases of the 3′-end nucleotide. More advantageously, the interrogation position of the allele-specific HCP oligonucleotide is within about 6 bases of the 3′-end nucleotide of the allele-specific HCP oligonucleotide. In another preferred embodiment, the interrogation position of the allele-specific HCP oligonucleotide is located within positions 4 and 6 from the 3′-end nucleotide. Most preferably, the interrogation position of the allele-specific HCP oligonucleotide is located in position 5 from the 3′-end nucleotide.

According to a preferred embodiment, the first oligonucleotide used in step (a) has at its first hybridization portion at least one artificial mismatch nucleotide substantially adjacent the interrogation position of the first oligonucleotide in which the mismatch nucleotide comprises an universal base or non-discriminatory analog base.

It is preferable that the first hybridization portion of the HCP oligonucleotide used in step (a) contains at least 6 nucleotides in length, which is a minimal requirement of length for hybridization. More preferably, the first hybridization portion is about 8 to 30 nucleotides in length. Most preferably, the first hybridization portion is about 10 to 15 nucleotides in length.

The term “interrogation position” as used herein refers to the location of a specific nucleotide base of interest within a target nucleic acid. For example, in the analysis of SNPs, the “interrogation position” in the target nucleic acid is in position what would be different from wild type. The interrogation position also includes the location of nucleotide sequence of an oligonucleotide that is complementary to an interrogation position of the target nucleic acid. The interrogation position of the target nucleic acid is opposite the interrogation position of the oligonucleotide, when the oligonucleotide hybridized with the target nucleic acid.

In still further aspect of this invention, there is provided a kit for identifying a nucleotide variation in a target nucleic acid, which comprises the oligonucleotide or oligonucleotide set (including the first and the second oligonucleotides) described above. The present kit may optionally include the reagents required for hybridization reaction such as buffers. Optimal amounts of reagents to be used in a given reaction can be readily determined by the skilled artisan having the benefit of the current disclosure. The kits, typically, are adapted to contain in separate packaging or compartments the constituents afore-described.

The following specific examples are intended to be illustrative of the invention and should not be construed as limiting the scope of the invention as defined by appended claims.

### EXAMPLES

In the experimental disclosure which follows, the following abbreviations apply: M (molar), mM (millimolar), μM (micromolar), g (gram), μg (micrograms), ng (nanograms), l (liters), ml (milliliters), μl (microliters), ° C. (degree Centigrade), Promega (Promega Co., Madison, USA), Roche (Roche Diagnostics, Mannheim, Germany), QIAGEN (QIAGEN GmbH, Hilden, Germany), Bio-Rad (Bio-Rad Laboratories, Hercules, USA), Applied Biosystems (Foster City, Calif., USA), Amersham (Amersham Phamacia Biotech, Piscataway, USA), and Stratagene (Stratagene, La Jolla, USA).

The oligonucleotide sequences used in the Examples are shown in Table 1.

### Example 1

### Effect of Universal Base Residues in HPC Oligonucleotide on Hybridization Specificity

The effect of universal base residues such as deoxyinosines positioned between the 3′- and 5′-end portions of HPC oligonucleotide was evaluated by SNP genotyping analysis using three different types of oligonucleotides each having allele-specific 10-mers, including conventional short and long oligonucleotides and HPC oligonucleotide.

A. Synthesis of Oligonucleotides

Oligonucleotides used in the Example 1 having sequences shown in Table 1 were synthesized by means of a DNA synthesizer (Expedite 8900 Nucleic Acid Synthesis System, Applied Biosystems (ABI)) according to a standard protocol. In the HPC oligonucleotides, deoxyinosine was incorporated using deoxyinosine CE phosphoramidite (ABI). The oligonucleotides were purified by means of an OPC cartridge (ABI), and their concentrations were determined by UV spectrophotometry at 260 nm. In the HPC oligonucleotides described below, “I” symbolizes deoxyinosine.

The allele-specific conventional short oligonucleotides for detecting a SNP in exon 4 of the TP53 gene are as follows:

| | | |
|---|---|---|
| P53NA | 5′-TCCCCGCGTG-3′ | (SEQ ID NO: 1) |
| and | | |
| P53NB | 5′-TCCCCCCGTG-3′. | (SEQ ID NO: 2) |

The allele-specific conventional long oligonucleotides for detecting a SNP in exon 4 of the TP53 gene are as follows:

(SEQ ID NO: 3)

P53NA-JYC7
5′-GTCTACCAGGCATTCGCTTTGCTCCCCGCGTG-3′
and

(SEQ ID NO: 4)

P53NB-JYC7
5′-GTCTACCAGGCATTCGCTTTGCTCCCCCCGTG-3′.

The polymorphic base is underlined and the position of the polymorphic base is considered as an interrogation position. The interrogation position is placed at the center of the allele-specific nucleotide sequence.

The 5′-end portion of the allele-specific conventional long and HPC oligonucleotides is a second hybridization portion comprising a pre-selected arbitrary nucleotide sequence and serves as a universal probing site for the second hybridization. The second hybridization portion sequence is:

JYC7 5′-GTCTACCAGGCATTCGCTTTGC-3′′ (SEQ ID NO:7). The oligonucleotide sequence complementary to the second hybridization portion sequence of the allele-specific conventional long and HPC oligonucleotides is:

| | |
|---|---|
| JYC7R | |
| 5′-GCAAAGCGAATGCCTGGTAGAC-3′′. | (SEQ ID NO: 8) |

B. Immobilization of the Oligonucleotides

Each of the conventional short and long oligonucleotides and HPC oligonucleotides was immobilized on a nylon membrane in accordance with the following manner. The oligonucleotides (6 μl of 100 μM) were spotted on a Hybond-N+ membrane (Amersham Pharmacia Biotech) by using a dot blotting device (Bio-Rad) or a pipetting. The obtained membrane was allowed to be dried and fixed by using an optimized UV crosslinking procedure.

C. DNA Sample Preparation

A region containing a single nucleotide polymorphism (SNP) of human p53 (TP53) gene (Matlashewsli et al., 1987; Lamb and Crawford, 1986) was amplified using either conventional primer system or annealing control primer system. The annealing control primer has been developed by the present inventor and disclosed in PCT/KR02/01781. DNA templates were obtained from human blood samples that have a SNP in exon 4 of the TP53 gene (Joseph Sambrook et al., Molecular Cloning, A Laboratory Manual, Cold Spring Harbor Laboratory Press, Cold Spring Harbor, N.Y. (2001)). This polymorphism is expressed as an Arg→Pro substitution at amino acid position 72 by replacing G with C. A 349 nucleotide sequence between nucleotide 11991 and 12339 of the TP53 gene was amplified from mutant-type homozygotes of templates by a set of the following primers:

| | | |
|---|---|---|
| P53N-ACP | 5′-TATGAATGCTGTGACGGCCGAIIIIICCTCTGACTGCTCTTTTCAC-3′ | (SEQ ID NO: 9) |
| and | | |
| P53C-ACP | 5′-TCACAGAAGTATGCCAAGCGAIIIIIATTGAAGTCTCATGGAAGCC-3′. | (SEQ ID NO: 10) |

The amplification reaction mixture in a final volume of 49.5 μl containing 50 ng of the genomic DNA containing the SNP in exon 4 of the TP53 gene, 5 μl of 10×PCR reaction buffer (Promega), 5 μl of 25 mM MgCl₂, 5 μl of dNTP (2 mM each dATP, dCTP, dGTP and dTTP), 1 μl of P53N-ACP primer (10 μM) and 1 μl of P53C-ACP primer (10 μM) was pre-heated at 94° C., while holding the tube containing the reaction mixture at the 94° C., 0.5 μl of Taq polymerase (5 units/μl, Promega) was added into the reaction mixture, and then the PCR reactions was performed as follows: 30 cycles of 94° C. for 40 sec, 65° C. for 40 sec, and 72° C. for 40 sec; followed by a 5 min final extension at 72° C. The amplified products containing the flanking region of the SNP was purified using a Qiagen (Chatsworth, Calif.) PCR purification kit.

D. Labeling of Sample DNA or Oligonucleotides

The amplified target genomic DNA segments were labeled with [α-³²P]dCTP (3,000 Ci/mmol, Amersham Phamacia Biotech) using the Random Prime DNA Labeling Kit (Roche) as known in the art (Multiprime DNA labeling systems booklet, “Amersham”, 1989). The oligonucleotide, JYC7R, which is complementary to the second hybridization portion sequence of the conventional long and BPC oligonucleotides was end-labeled with [γ-³²P]ATP (3,000 Ci/mmol, Amersham Phamacia Biotech) and T4 polynucleotide as known in the art Maxam & Gilbert, Methods in Enzymology, 65: 499-560(1986)).

E. Hybridization Reaction

(a) First Hybridization

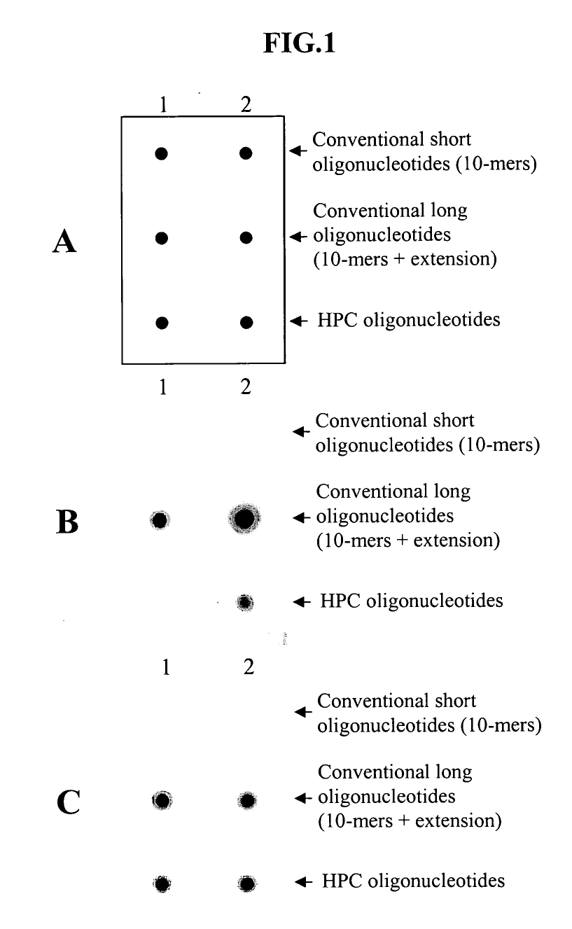

The first hybridization was conducted using the above-mentioned membrane filter on which the conventional and HPC oligonucleotides were immobilized and the radioactively labeled genomic DNA segment in 10 ml of Quick hybridization solution (Stratagene) at from 40° C. to 50° C. for 1 hour to 4 hours. After completion of hybridization, the hybrid was washed three to five times with a buffer solution of 2×SSC and 0.1% SDS at room temperature for 1 minute to 10 minutes, and air-dried. The membrane was exposed to Kodak X-Omat XK-1 film with a Fuji intensifying screen at −80° C. Subsequently, the radiation dose of each dot was measured by autoradiography to evaluate the strength of the hybridization (FIG. 1B).

(b) Second Hybridization

The resultant membrane filter used in the first hybridization was then re-used in the second hybridization without stripping for removal of the first probe used in the first hybridization. The second hybridization was conducted using the first hybridized filter and the radioactively labeled oligonucleotide, JYC7R, comprising a nucleotide sequence complementary to the second hybridization portion sequence of the conventional and HPC oligonucleotides in 10 ml of Quick hybridization solution (Stratagene) at from 50° C. to 65° C. for 1 hour to 4 hours. After completion of hybridization, the hybrid was washed three times with a buffer solution of 2×SSC and 0.1% SDS at from 50° C. to 65° C. for 10 minute to 1 hour, and air-dried. The membrane was exposed to Kodak X-Omat XK-1 film with a Fuji intensifying screen at −80° C. Subsequently, the radiation dose of each dot was measured by autoradiography to evaluate the strength of the hybridization (FIG. 1C).

FIG. 1A indicates that the conventional and HPC oligonucleotides were spotted equally on each membrane. Lanes 1 and 2 represent the allele-specific conventional or HPC oligonucleotides for wild-type [P53NA (SEQ ID NO:1), P53NA-JYC7 (SEQ ID NO:3), and P53NA-HPC (SEQ ID NO:5)] and mutant-type [P53NB (SEQ ID NO:2), P53NB-JYC7 (SEQ ID NO:4), and P53NB-HPC (SEQ ID NO:6)], respectively.

FIG. 1B shows the results of the first hybridization for the mutant-type genomic DNA fragment obtained from the step C. Under the first hybridization conditions, no signal was detected for the conventional short oligonucleotide. The mutant-type genomic DNA fragment containing mutant-type genotype was hybridized to both the wild-type (lane 1) and mutant-type (lane 2) allele-specific conventional long oligonucleotides. In contrast, the mutant-type genomic DNA fragment containing mutant-type genotype was hybridized to the mutant-type allele-specific HPC oligonucleotide (lane 2) but not to the wild-type allele-specific HPC oligonucleotide (lane 1).

From these results, the conventional short oligouncleotide could not even form hybrids under such high stringency condition whereas the conventional long and HPC oligonucleotides form hybrids under such conditions, which means that additional sequences such as the second hybridization portion sequence increases the hybridization strength (efficiency) of their first hybridization portions. However, the first hybridization portion of the conventional long oligonucleotides was not sensitive enough to discriminate one base nucleotide mismatch because the second hybridization portion sequence may be partially involved in the first hybridization. On the other hand, it is more clear that the presence of an universal base residue group forming a boundary between the first and second hybridization portions restricts a hybridization portion to the first hybridization portion under the first hybridization conditions which are relatively high stringency conditions so that the second hybridization portion may be completely excluded from the hybridization reaction, such that the first hybridization portion is hybridized specifically to its target nucleotide sequence.

FIG. 1C showed the results of the second hybridization using the radioactively labeled oligonucleotide, JYC7R, comprising a nucleotide sequence complementary to the second hybridization portion sequence of the HPC and conventional long oligonucleotides.

The results showed that each spot has almost equal radioactivity. The equal radioactivity indicates that any artificial factor, such as the failures of spotting, immobilization and establishment of optimal hybridization conditions, did not affect on the first hybridization and also the allele-specific HPC oligonucleotides worked properly as a probe for the first hybridization in each experiment, thereby the second hybridization verified the results of the first hybridization.

### Example 2

### SNP Genotyping Using HPC Oligonucleotides

In order to demonstrate the application of HPC oligonucleotides to single nucleotide polymorphism genotyping, HPC oligonucleotides have been applied for a single nucleotide polymorphism (SNP) of human p53 (TP53) gene.

The allele-specific HPC oligonucleotides for detecting a SNP in exon 4 of the TP53 gene are as follows:

| | | |
|---|---|---|
| P53N1A-HPC | 5′-GTCTACCAGGCATTCGCTTCATIIIIICCCCGCGTGG-3′, | (SEQ ID NO: 11) |
| P53N1B-HPC | 5′-GTCTACCAGGCATTCGCTTCATIIIIICCCCCCGTGG-3′, | (SEQ ID NO: 12) |
| P53N2A-HPC | 5′-GTCTACCAGGCATTCGCTTCATIIIIITCCCCGCGTG-3′, | (SEQ ID NO: 13) |
| P53N2B-HPC | 5′-GTCTACCAGGCATTCGCTTCATIIIIITCCCCCCGTG-3′, | (SEQ ID NO: 14) |
| P53N3A-HPC | 5′-GTCTACCAGGCATTCGCTTCATIIIIICTCCCCGCGT-3′, | (SEQ ID NO: 15) |
| P53N3B-HPC | 5′-GTCTACCAGGCATTCGCTTCATIIIIICTCCCCCCGT-3′, | (SEQ ID NO: 16) |
| P53N4A-HPC | 5′-GTCTACCAGGCATTCGCTTCATIIIIIGCTCCCCGCG-3′, | (SEQ ID NO: 17) |
| P53N4B-HPC | 5′-GTCTACCAGGCATTCGCTTCATIIIIIGCTCCCCCCG-3′, | (SEQ ID NO: 18) |
| P53N5A-HPC | 5′-GTCTACCAGGCATTCGCTTCATIIIIIGCTCCCCG-3′, | (SEQ ID NO: 19) |
| and | | |
| P53N5B-HPC | 5′-GTCTACCAGGCATTCGCTTCATIIIIIGCTCCCCC-3′. | (SEQ ID NO: 20) |

In the HPC oligonucleotides described above, “I” symbolizes deoxyinosine. The polymorphic base is underlined at the first hybridization (3′-end) portion of each allele-specific HPC oligonucleotide and the position of the polymorphic base is considered as an interrogation position. The interrogation position is placed at several different positions from the 3′-end of allele-specific HPC oligonucleotides in order to determine the most critical position in hybridization specificity for detecting the SNP.

The 5′ nd portion of the allele-specific HPC oligonucleotides is a second hybridization portion comprising a pre-selected arbitrary nucleotide sequence and serves as a universal probing site for the second hybridization. The second hybridization portion sequence is:

| | |
|---|---|
| JYC4 | |
| 5′-GTCTACCAGGCATTCGCTTCAT-3′JYC2. | (SEQ ID NO: 21) |

The oligonucleotide sequence complementary to the second hybridization portion sequence of the allele-specific HPC oligonucleotides is:

| | |
|---|---|
| JYC4R | |
| 5′-ATGAAGCGAATGCCTGGTAGTC-3′. | (SEQ ID NO: 22) |

The allele-specific HPC oligonucleotides were immobilized on a nylon membrane as described in the Example 1. The region containing a SNP in exon 4 of human p53 (TP53) gene (Matlashewsli et al., 1987; Lamb and Crawford, 1986) was amplified as described in the Example 1 using the same primers used in the step C of Example 1.

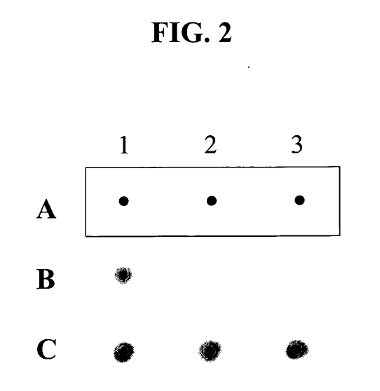

The amplified target genomic DNA segments were labeled with [α-³²P]dCTP (3,000 Ci/mmol, Amersham Phamacia Biotech) as described in the Example 1. The oligonucleotide, JYC4R, which is complementary to the second hybridization portion sequence of the allele-specific HPC oligonucleotides was end-labeled with [γ-³²P]ATP (3,000 Ci/mmol, Amersham Phamacia Biotech) and T4 polynucleotide as described in the Example 1. The first and second hybridizations were conducted as described in the Example 1. The radiation dose of each dot was measured by autoradiography to evaluate the strength of the hybridization (FIGS. 2-4).

FIG. 2B shows the results of the first hybridization for the wild-type genomic DNA fragment. The wild-type genomic DNA fragment containing a wild-type genotype was hybridized to the wild-type allele-specific HPC oligonucleotide (lane 1) but neither to the mutant-type allele-specific HPC oligonucleotide (lane 2) nor to the negative control HPC oligonucleotide (lane 3). In addition, FIG. 2C showed the results of the second hybridization using the radioactively labeled oligonucleotide, JYC4R, comprising a nucleotide sequence complementary to the second hybridization portion sequence of the allele-specific HPC oligonucleotides. The results showed that each spot has almost equal radioactivity. The equal radioactivity indicates that any artificial factor, such as the failures of spotting, immobilization and establishment of optimal hybridization conditions, did not affect on the first hybridization and also the allele-specific HPC oligonucleotides worked properly as a probe for the first hybridization in each experiment, thereby the second hybridization verified the results of the first hybridization. Thus, these results demonstrate that the allele-specific HPC oligonucleotides are highly sensitive enough to detect even a single-base mismatching.

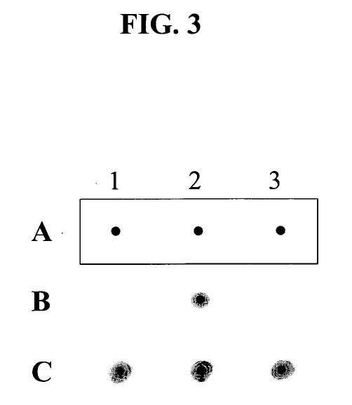

FIG. 3B shows the results of the first hybridization for the mutant-type genomic DNA fragment. In contrast to FIG. 2B, the mutant-type genomic DNA fragment containing a mutant-type genotype was hybridized to the mutant-type allele-specific HPC oligonucleotide (ane 2) but not to the wild-type allele-specific HPC oligonucleotide (lane 1).

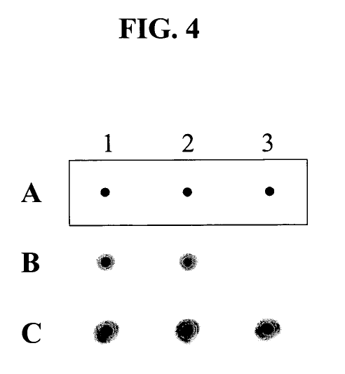

FIG. 4B shows the results of the first hybridization for the heterozygous-type genomic DNA fragment. The heterozygous-type genomic DNA fragment containing a heterozygous genotype was hybridized to both wild-type (lane 1) and mutant-type allele-specific HPC oligonucleotides (lane 2).

FIGS. 3C and 4C show the results of the second hybridization to verify the results of the first hybridizations for the mutant-type and heterozygous-type genotyping, respectively. These results are the same as those of FIG. 2C.

Moreover, it was found that when the allele-specific HPC oligonucleotides have an interrogation position at the center of the first hybridization portion sequence, the hybridization specificity is most successfully accomplished.

In view of these results, the HPC oligonucleotides of the subject invention allows us to discriminate between target sequences that differ by as little as a single nucleotide without adjusting the length, position, and strand specificity of oligonucleotide probes, or varying the amount applied to membrane so that SNP genotyping analysis using the HPC oligonucleotides was performed in an easy and economic as well as reliable manner. Furthermore, it is also expected that speed and efficiency will be greatly improved when multiple SNP genotyping is achieved using the HPC oligonucleotides.

TABLE 1

| | | | |
|---|---|---|---|
| SEQ | | | +L,39 |
| ID NO | Designation | Sequence Information | |

| | | |
|---|---|---|
| 1 | P53NA | 5′-TCCCCGCGTG-3′ |
| 2 | P53NB | 5′-TCCCCCCGTG-3′ |
| 3 | P53NA-JYC7 | 5′-GTCTACCAGGCATTCGCTTTGCTCCCCGCGTG-3′ |
| 4 | P53NB-JYC7 | 5′-GTCTACCAGGCATTCGCTTTGCTCCCCCCGTG-3′ |
| 5 | P53NA-HPC | 5′-GTCTACCAGGCATTCGCTTTGCIIIIITCCCCGCGTG-3′ |
| 6 | P53NB-HPC | 5′-GTCTACCAGGCATTCGCTTTGCIIIIITCCCCCCGTG-3′ |
| 7 | JYC7 | 5′-GTCTACCAGGCATTCGCTTTGG-3′ |
| 8 | JYC7R | 5′-GCAAAGCGAATGCCTGGTAGAC-3′ |
| 9 | P53N-ACP | 5′-TATGAATGCTGTGACGCCGAIIIIICCTCTGACTGCTC |
| | | TTTTCAC-3′ |
| 10 | P53C-ACP | 5′-TCACAGAAGTATGCCAAGCGAIIIIIATTGAAGTCTCAT |
| | | GGAAGCC-3′ |
| 11 | P53N1A-HPC | 5′-GTCTACCAGGCATTCGCTTCATIIIIICCCCGCGTGG-3′ |
| 12 | P53N1B-HPC | 5′-GTCTACCAGGCATTCGCTTCATIIIIICCCCCCGTGG-3′ |
| 13 | P53N2A-HPC | 5′-GTCTACCAGGCATTCGCTTCATIIIIITCCCCGCGTG-3′ |
| 14 | P53N2B-HPC | 5′-GTCTACCAGGCATTCGCTTCATIIIIITCCCCCCGTG-3′ |
| 15 | P53N3A-HPC | 5′-GTCTACCAGGCATTCGCTTCATIIIIICTCCCCGCGT-3′ |
| 16 | P53N3B-HPC | 5′-GTCTACCAGGCATTCGCTTCATIIIIICTCCCCCCGT-3′ |
| 17 | P53N4A-HPC | 5′-GTCTACCAGGCATTCGCTTCATIIIIIGCTCCCCGCG-3′ |
| 18 | P53N4B-HPC | 5′-GTCTACCAGGCATTCGCTTCATIIIIIGCTCCCCCCG-3′ |
| 19 | P53N5A-HPC | 5′-GTCTACCAGGCATTCGCTTCATIIIIIGCTCCCCG-3′ |
| 20 | P53N5B-HPC | 5′-GTCTACCAGGCATTCGCTTCATIIIIIGCTCCCCC-3′ |
| 21 | JYC4 | 5′-GTCTACCAGGCATTCGCTTCAT-3′ |
| 22 | JYC4R | 5′-ATGAAGCGAATGCCTGGTAGTC-3′ |

I is deoxyinosine

### REFERENCES

Azhikina, T., Veselovskaya, S., Myasnikov, V., Ermolayeva, O., Sverdlov, E. (1993) Strings of contiguous modified pentanucleotides with increased DNA-binding affinity can be used for DNA sequencing by primer walking. Proc. Natl. Acad. Sci. USA 90, 11460-11462.
Breslauer, K. J., Frank R., Blocker, H., Marky, L. A. (1986) Predicting DNA duplex stability from the base sequence. Proc. Natl. Acad. Sci. USA 83, 3746-3750.
Conner, B. J., Reyes, A. A., Morin, C., Itakura, K., Teplitz, R. L., Wallace, R. B. (1983) Detection of sickle cell α-globin allele by hybridization with synthetic oligonucleotides. Proc. Natl. Acad. Sci. USA 80, 278-282.
Doktycz, M. J., Morris, M. D., Dormady, S. J., Beattie, K. L., Jacobson, K. B. (1995) Optical melting of 128 octamer DNA duplexes effects of base pair location and nearest neighbors on thermal stability. J. Biol. Chem. 270, 8439-8445.
Drmanac, R., Strezoska, Z., Labat, I., Drmanac, S., Crkvenjakov, R. (1990) Reliable hybridization of oligonucleotides as short as six nucleotides. DNA Cell Biol. 9, 527-534.
Guo, Z., Liu, Q., Smith, L. M. (1997) Enhanced discrimination of single nucleotide polymorphisms by artificial mismatch hybridization. Nat Biotechnol. 15, 331-335.
Ikuta, S., Takagi, K., Wallace, B. R., Itakura, K. (1987) Dissociation kinetics of 19 base paired oligonucletotide-DNA duplexes containing different single mismatch base pair. Nucleic Acids Res. 15, 797-811.
Lamb, P., Crawford, L. (1986) Characterization of the human p53 gene. Mol Cell Biol. 6, 1379-1385.
Matlashewski, G. J., Tuck, S., Pim, D., Lamb, P., Schneider, J., Crawford, L. V. (1987) Primary structure polymorphism at amino acid residue 72 of human p53. Mol Cell Biol. 7, 961-963.
McGraw, R. A., Steffe, E. K., Baxter, S. M. (1990) Sequence-dependent oligonucleotide-target duplex stabilities: rules from empirical studies with a set of twenty-mers. BioTechniques 8, 674-678.
Saiki, R. K., Walsh, P. S., Levenson, C. H., Erlich, H. A. (1989) Genetic analysis of amplified DNA with immobilized sequence-specific oligonucleotide probes. Proc. Natl. Acad. Sci. USA 86, 6230-6234.
Tibanyenda, N., De Bruin, S. H., Haasnoot, C. A. G., Van der Marel, G. A., Van Boom, J. H., Hilbers, C. W. (1984) The effect of single base-pair mismatches on the duplex stability of d(TATTAATATCAAGTTG). d(CAACTTGATATTAATA). Eur. J. Biochem. 139, 19-27.
Wallace, B. R., Johnson, M. J., Hirose, T., Miyake, T., Kawashima, E. H., Itakura, K. (1981) The use of synthetic oligonucleotides as hybridization probes. Hybridization of oligonucleotides of mixed sequence to rabbit β-globin DNA. Nucleic Acids Res. 9, 879-894.
Werntges, H., Steger, G., Riesner, D., Fritz, H.-J. (1986) Mismatches in DNA double strands: thermodynamic parameters and their correlation to repair differences. Nucleic Acid. Res. 14, 3773-3790.

1. An oligonucleotide probe for analyzing a target nucleotide sequence in a sample nucleic acid by hybridization, said oligonucleotide comprising the following structure:
5′-Xₚ—Y_{q}-Zᵣ-3′ or 5′-Zᵣ-Y_{q}—Xₚ-3′
wherein Xₚ represents a first hybridization portion having a specific hybridizing nucleotide sequence substantially complementary to said target nucleotide sequence in said sample nucleic acid to hybridize therewith; Y_{q} represents a regulator portion comprising at least two universal bases or non-discriminatory base analogs; Zᵣ represents a second hybridization portion having a pre-selected arbitrary nucleotide sequence; p, q and r represent the number of nucleotides; and X, Y and Z is deoxyribonucleotide or ribonucleotide.

2. The oligonucleotide probe according to claim 1, which is involved in two individual hybridizations as a first and second hybridizations, wherein said first hybridization portion is used as a specific hybridization site at a first hybridization, and said second hybridization portion serves as a universal hybridizing site at a second hybridization.

3. The oligonucleotide probe according to claim 1, wherein said regulator portion is capable of controlling a hybridization portion of said oligonucleotide.

4. The oligonucleotide probe according to claim 1, wherein said universal base or non-discriminatory base analog forms base-pairs with each of the natural DNA/RNA bases with little discrimination between said natural DNA/RNA bases.

5. The oligonucleotide probe according to claim 4, wherein said universal bases or non-discriminatory base analogs are selected from the group consisting of deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitroindole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4nitrobenzimidazole, phosphoramidate-3-nitropyrrole, 2′-0-methoxyethyl inosine, 2′0-methoxyethyl nebularine, 2′-0-methoxyethyl 5-nitroindole, 2′-0-methoxyethyl 4-nitro-benzimidazole, 2′-O-methoxyethyl 3-nitropyrrole, and combinations thereof.

6. The oligonucleotide probe according to claim 5, wherein said universal bases or non-discriminatory base analogs are selected from the group consisting of deoxyinosine, 1-(2′-deoxy-beta-D-ribofuranosyl)-3-nitropyrrole and 5-nitroindole.

7. The oligonucleotide probe according to claim 1, wherein said regulator portion comprises contiguous universal bases or non-discriminatory base analogs.

8. The oligonucleotide probe according to claim 1, wherein said deoxyribonucleotide is naturally occurring dNMP, modified nucleotide and non-natural nucleotide.

9. The oligonucleotide probe according to claim 1, wherein p represents an integer of 6 to 100.

10. The oligonucleotide probe according to claim 1, wherein q is at least 3.

11. The oligonucleotide probe according to claim 10, wherein q is at least 4.

12. The oligonucleotide probe according to claim 10, wherein q represents an integer of 2 to 15.

13. The oligonucleotide probe according to claim 1, wherein r represents an integer of 15 to 100.

14. The oligonucleotide probe according to claim 1, wherein the second hybridization portion has a pre-selected arbitrary nucleotide sequence substantially not complementary to any site on said sample nucleic acid

15. The oligonucleotide probe according to claim 1, which is immobilized on an insoluble carrier.

16. A kit for carrying out a hybridization, wherein said kit comprises an oligonucleotide according to claim 1 and optionally, a hybridization reagent.

17. A method for detecting the presence of a target nucleotide sequence in a sample nucleic acid by hybridization, wherein said method comprises the steps of:
(a) performing a first hybridization using a first oligonucleotide according to claim 1 having at its first hybridization portion a specific hybridizing nucleotide sequence substantially complementary to said target nucleotide sequence to hybridize therewith under conditions in which said first hybridization portion of said first oligonucleotide is to be hybridized to said target nucleotide sequence; and
(b) detecting the presence or absence of said target nucleotide sequence substantially complementary to said first hybridization portion of said first oligonucleotide in said sample nucleic acid through a signal indicative of the hybridization between said target nucleotide sequence and said first hybridization portion.

18. The method according to claim 17, wherein the method further comprises the steps of:
(c) performing a second hybridization using a second oligonucleotide having a nucleotide sequence substantially complementary to said second hybridization portion of said first oligonucleotide used in step (a) to hybridize therewith under conditions in which said second oligonucleotide is to be hybridized to said second hybridization portion sequence of said first oligonucleotide; and
(d) detecting a signal indicative of the hybridization between said second hybridization portion of said first oligonucleotide and said second oligonucleotide, so that the presence or absence of said signal of step (b) is confirmed to be ascribed solely to the hybridization between said target nucleotide sequence and said first hybridization portion of said first oligonucleotide.

19. A method for identifying a nucleotide variation in a target nucleotide sequence of a sample nucleic acid, wherein said method comprises the steps of:
(a) performing a first hybridization using a first oligonucleotide of claim 1 having at its first hybridization portion a specific hybridizing nucleotide sequence substantially complementary to said target nucleotide sequence of said sample nucleic acid to hybridize therewith under conditions in which said first hybridization portion of said first oligonucleotide is to be hybridized to said target nucleotide sequence of said sample nucleic acid, wherein each of said first oligonucleotide and said target nucleotide sequence comprises an interrogation position corresponding to said nucleotide variation, whereby said first oligonucleotide including said nucleotide variation is hybridized to said target nucleotide sequence when said interrogation position is occupied by the complementary nucleotide of said first oligonucleotide to its corresponding nucleotide of said target nucleotide sequence; and
(b) identifying said nucleotide variation in said target nucleotide sequence of said sample nucleic acid by detecting a signal indicative of the hybridization between said target nucleotide sequence and said first hybridization portion of said first oligonucleotide.

20. The method according to claim 19, wherein the method further comprises the steps of:
(c) performing a second hybridization using a second oligonucleotide having a nucleotide sequence substantially complementary to said second hybridization portion of said first oligonucleotide used in the step (a) to hybridize therewith under conditions in which said second oligonucleotide is to be hybridized with said second hybridization portion sequence of said first oligonucleotide; and
(d) detecting a signal indicative of the hybridization between said second hybridization portion of said first oligonucleotide and said second oligonucleotide, so that the presence or absence of said signal of step (b) is confirmed to be ascribed solely to the hybridization between said target nucleotide sequence and said first hybridization portion of said first oligonucleotide.

21. The method according to claim 17, wherein said regulator portion of said first oligonucleotide restricts a hybridization portion of said first oligonucleotide with said target nucleotide sequence to said first hybridization portion.

22. The method according to claim 17, wherein said regulator portion of said first oligonucleotide enhances a hybridization specificity of said first hybridization portion of said first oligonucleotide.

23. The method according to claim 19, wherein said sample nucleic acid is a short nucleotide segment including a nucleotide variation which is prepared by amplifying the corresponding nucleotide sequence of said short nucleotide segment.

24. The method according to claim 19, wherein said sample nucleic acid is more than one target short nucleotide segment each including a nucleotide variation which is prepared by amplifying each corresponding nucleotide sequence of more than one short nucleotide segment.

25. The method according to claim 19, wherein said nucleotide variation is single nucleotide polymorphism or point mutation.

26. The method according to claim 19, wherein said nucleotide variation is contained within human nucleic acid.

27. The method according to claim 19, wherein said nucleotide variation is contained within nucleic acid of an organism that can cause an infectious disease.

28. The method according to claim 19, wherein said first hybridization portion of said first oligonucleotide used in step (a) comprises an interrogation position occupied by a complementary nucleotide to the corresponding nucleotide which corresponds to a nucleotide variation.

29. The method according to claim 19, wherein said interrogation position of said first oligonucleotide used in step (a) is in the center of its first hybridization portion.

30. The method according to claim 19, wherein said first hybridization portion of said first oligonucleotide used in step (a) is 8 to 30 nucleotides in length.

31. The method according to claim 30, wherein said first hybridization portion of said first oligonucleotide used in step (a) is 10 to 15 nucleotides in length.

32. The method according to claim 19, wherein said interrogation position of said first oligonucleotide used in step (a) is within about 10 bases of the 3′-end nucleotide of said first oligonucleotide.

33. The method according to claim 32, wherein said interrogation position of said first oligonucleotide used in step (a) is within about 6 bases of the 3′-end nucleotide of said first oligonucleotide.

34. The method according to claim 33, wherein said interrogation position of said first oligonucleotide used in step (a) is located within positions 4 and 6 from the 3′-end nucleotide of said first oligonucleotide.

35. The method according to claim 19, wherein said first oligonucleotide used in step (a) has at its first hybridization portion at least one artificial mismatch nucleotide substantially adjacent said interrogation position of said first oligonucleotide in which said mismatch nucleotide comprises an universal base or non-discriminatory analog base.

36. A kit for identifying a nucleotide variation in a target nucleic acid of a sample nucleic acid, which comprises the oligonucleotide or oligonucleotide set indicated in claim 19.


SEQUENCE LISTING (24 sequences)

SEQ ID NO: 1 - DNA, 10 bp, Artificial Sequence
  Synthetic polynucleotide sequence
tccccgcgtg

SEQ ID NO: 2 - DNA, 10 bp, Artificial Sequence
  Synthetic polynucleotide sequence
tccccccgtg

SEQ ID NO: 3 - DNA, 32 bp, Artificial Sequence
  Synthetic polynucleotide sequence
gtctaccaggcattcgctttgctccccgcgtg

SEQ ID NO: 4 - DNA, 32 bp, Artificial Sequence
  Synthetic polynucleotide sequence
gtctaccaggcattcgctttgctccccccgtg

SEQ ID NO: 5 - DNA, 37 bp, Artificial Sequence
  Synthetic polynucleotide sequence
gtctaccaggcattcgctttgcnnnnntccccgcgtg

SEQ ID NO: 6 - DNA, 37 bp, Artificial Sequence
  Synthetic polynucleotide sequence
gtctaccaggcattcgctttgcnnnnntccccccgtg

SEQ ID NO: 7 - DNA, 22 bp, Artificial Sequence
  Synthetic polynucleotide sequence
gtctaccaggcattcgctttgc

SEQ ID NO: 8 - DNA, 22 bp, Artificial Sequence
  Synthetic polynucleotide sequence
gcaaagcgaatgcctggtagac

SEQ ID NO: 9 - DNA, 45 bp, Artificial Sequence
  Synthetic polynucleotide sequence
tatgaatgctgtgacgccgannnnncctctgactgctcttttcac

SEQ ID NO: 10 - DNA, 46 bp, Artificial Sequence
  Synthetic polynucleotide sequence
tcacagaagtatgccaagcgannnnnattgaagtctcatggaagcc

SEQ ID NO: 11 - DNA, 37 bp, Artificial Sequence
  Synthetic polynucleotide sequence
gtctaccaggcattcgcttcatnnnnnccccgcgtgg

SEQ ID NO: 12 - DNA, 37 bp, Artificial Sequence
  Synthetic polynucleotide sequence
gtctaccaggcattcgcttcatnnnnnccccccgtgg

SEQ ID NO: 13 - DNA, 37 bp, Artificial Sequence
  Synthetic polynucleotide sequence
gtctaccaggcattcgcttcatnnnnntccccgcgtg

SEQ ID NO: 14 - DNA, 37 bp, Artificial Sequence
  Synthetic polynucleotide sequence
gtctaccaggcattcgcttcatnnnnntccccccgtg

SEQ ID NO: 15 - DNA, 37 bp, Artificial Sequence
  Synthetic polynucleotide sequence
gtctaccaggcattcgcttcatnnnnnctccccgcgt

SEQ ID NO: 16 - DNA, 37 bp, Artificial Sequence
  Synthetic polynucleotide sequence
gtctaccaggcattcgcttcatnnnnnctccccccgt

SEQ ID NO: 17 - DNA, 37 bp, Artificial Sequence
  Synthetic polynucleotide sequence
gtctaccaggcattcgcttcatnnnnngctccccgcg

SEQ ID NO: 18 - DNA, 37 bp, Artificial Sequence
  Synthetic polynucleotide sequence
gtctaccaggcattcgcttcatnnnnngctccccccg

SEQ ID NO: 19 - DNA, 35 bp, Artificial Sequence
  Synthetic polynucleotide sequence
gtctaccaggcattcgcttcatnnnnngctccccg

SEQ ID NO: 20 - DNA, 35 bp, Artificial Sequence
  Synthetic polynucleotide sequence
gtctaccaggcattcgcttcatnnnnngctccccc

SEQ ID NO: 21 - DNA, 22 bp, Artificial Sequence
  Synthetic polynucleotide sequence
gtctaccaggcattcgcttcat

SEQ ID NO: 22 - DNA, 22 bp, Artificial Sequence
  Synthetic polynucleotide sequence
atgaagcgaatgcctggtagtc

SEQ ID NO: 23 - DNA, 16 bp, Artificial Sequence
  Synthetic polynucleotide sequence
tattaatatcaagttg

SEQ ID NO: 24 - DNA, 16 bp, Artificial Sequence
  Synthetic polynucleotide sequence
caacttgatattaata